(19) United States
(12) Patent Application Publication
(10) Pub. No.: US 2009/0215109 A1
(43) Pub. Date: Aug. 27, 2009

(54) APPARATUS AND METHOD FOR MEASURING LUMINESCENCE AND FLUORESCENCE OF TRANSFECTED CELLS OR ORGAN PARTS

(75) Inventor: Manfred HENNECKE, Remshalden (DE)

Correspondence Address: BROWDY AND NEIMARK, P.L.L.C.;624 NINTH STREET, NW, SUITE 300, WASHINGTON, DC, 20001-5303, US

(73) Assignee: Berthold Technologies GmbH & Co.KG, Bad Wildbad (DE)

(21) Appl. No.: 12/389,880

(22) Filed: Feb. 20, 2009

(30) Foreign Application Priority Data
Feb. 21, 2008 (DE) 102008010436.1

(51) Int. Cl. C12Q 1/02; C12M 1/00

(52) U.S. Cl. 435/29; 435/288.7

(57) ABSTRACT

An apparatus for measuring photon emissions from marked cells. The apparatus has the following components: a rotatable, at least partly transparent growth chamber/bioreactor (10) for accommodation of cell cultures, growth media and active substances; a light detector for detection of the photons emitted from the marked cells from the growth chamber (10); and a light-tight enclosure of at least the emission path of the photons from the growth chamber (10) to the light detector. The apparatus may be used for carrying out methods by which the effect of active substances on transfected cells is measured (FIG. 1).

### BACKGROUND OF THE INVENTION

To find and test new pharmaceutical drugs, for example for treating cancer, the effect of such drugs must be tested on living cells. Especially with cancer drugs, the objective often is to find substances that slow the growth of cancer cells or—ideally—kill the cancer cells. For this purpose cancer cells are specifically grown, induced to multiply and at a later point in time brought into contact with the substance that is to be tested. It is then observed how the substance that is to be tested affects the growth of the tumor cells.

Two basic methods for establishing the efficacy of such substances are known. In the first method, the transfected tumor cells are injected into living animals, usually mice, where, upon overpowering immune suppression, they form tumors. In the second method, the transfected tumor cells are grown in a nutrient solution by cell division. These two methods shall be examined first:

a) Method with Living Animals

Two sub-methods must be distinguished here:

In the first sub-method, the animal is killed after a certain period of time and a tissue histology is performed. This method has numerous shortcomings: on the one hand, a large number of animals in various stages of cancer is needed for a relevant finding. This is undesirable for ethical reasons alone. Furthermore, tissue histology is very time-consuming and labor-intensive. Because of the sections and stains, comparability does not always exist.

In the second sub-method, transfected cells are used. This is done by modifying bacterial DNA plasmids with luciferase genes or green fluorescence protein (GFP) genes and introducing them into tumor cells before these tumor cells are injected. After transcription and translation during the multiplication of the tumor cells, the gene sections are coded into proteins that are then present in the cells.

In the case of luciferase, photons are generated (bioluminescence) under addition of luciferin, adenosine triphosphate (ATP), oxygen, and calcium ions. The intensity of the bioluminescence is a measure for the number of tumor cells. In the case of GFP, the protein is excited with a certain wavelength (480 nm). The emitted light (520 nm) is measured (biofluorescence) and is likewise a measure for the number of tumor cells. Through photon detection of the bioluminescence or biofluorescence by means of photomultipliers or highly sensitive cameras, the emitted photons can be detected. The principle of the establishment of efficacy of a substance is based in the extent to which such substances (e.g. cytostatics) kill the cancer cells. Along with this cell death, the bioluminescence or biofluorescence is extinguished as well, because these proteins are no longer being reproduced. Typical intensity/time correlation curves are such that in the control group, the intensity continually increases; in the test group, however, it remains more or less constant or, in the best case scenario, decreases.

Use of the second sub-method significantly minimizes the number of animals that are needed. However, organs in the interior of the animals bodies are poorly detectable, as the photons are absorbed, for example, by hemoglobin or melamine. Liver damage, in particular, is very difficult to detect through in-vivo imaging, as this organ is particularly well supplied with blood. Moreover, in-vivo imaging is not automatable.

This bioluminescence or biofluorescence can be detected using suitable instruments, e.g. LB 940, LB 941, LB 960 or LB 983 marketed by Berthold Technologies GmbH & Co. KG.

b) Method without Living Animals

Here again, two sub-methods need to be distinguished, namely 2D methods and 3D methods. These methods are carried out, as a rule, with transfected cells that display bioluminescence or biofluorescence (see above).

In 2D methods, the tumor cells are grown either in microtiter plates or in other culture dishes. Here, there is no interference through hemoglobin, as the cells are grown without blood. The cells do have the tendency, however, to primarily settle at the bottom of the plate or dish, where they form a cell monolayer. The cells do not form any tissue-like three-dimensional cross-linkage with neighboring cells, which would be a prerequisite for a physiologically relevant finding. On the other hand, it is advantageous that the method can be automated to a high degree.

With 3D methods, so-called 3D growth chambers are used. In such instruments, a glass cylinder rotates about a horizontal axis. This rotation effects micro-gravitation forces, causing the cells to start to grow organ-like and to form three-dimensional cell clusters or organs. When this method is used, the following process steps are performed:
1. Introduction of transfected cells and of a nutrient solution.
2. To monitor the cell growth, manual aliquots are collected and the cell mass is determined.
3. Introduction of an active substance to be tested.
4. Repeated time-dependent manual removal of aliquots for measuring bioluminescence or biofluorescence.
5. Preparation of an intensity/time correlation from the preceding steps.

The shortcomings of this method relate to steps 2 and 4, as these are cumbersome, time-consuming, and not automatable, and are therefore labor-intensive and expensive.

### BRIEF SUMMARY OF THE INVENTION

The present invention provides improved apparatuses and methods for measuring luminescence or fluorescence in cells in such a way that a faster and resource-conserving measurement is attained.

More specifically, the present invention is embodied in an apparatus for measuring photon emissions from marked cells, which apparatus includes: a rotatable, at least partly transparent growth chamber/bioreactor for accommodation of cell cultures, growth media and active substances, the chamber/bioreactor comprising at least one feed device for introducing the marked cells and an active substance into the chamber/bioreactor; a light detector positioned for detection of photons emitted over an emission path from marked cells in the growth chamber/bioreactor; and a light-tight enclosure for at least the emission path for the photons from the growth chamber/bioreactor to the light detector.

The invention is further embodied in a method for emission measurement of transfected cells comprising the following process steps: providing the apparatus described above; introducing the transfected cells into the growth chamber/bioreactor; monitoring growth of the transfected cells in the growth chamber/bioreactor; introducing an active substance that is to be tested into the growth chamber/bioreactor; measuring photons emitted from the growth chamber/bioreactor, continually or in specifiable time intervals; and displaying and/or storing a representation of the time progression of the intensity of the measured photons.

Based on the known technology of 3D growth chambers (e.g. as marketed by Synthecon, Texas, USA) an apparatus for measuring light from transfected cells (reporter gene assay) is improved further; the underlying concept being the integration of the 3D growth chamber into the measuring apparatus.

It is therefore possible with the inventive apparatus to automatically and reproducibly test human cells or organ parts with, e.g., promoter-dependent DNA for their reaction to new pharmacological agents used as the active substances. Additionally, a dose-reaction correlation can be easily determined. Side effects, in particular on liver cells, can also be easily determined.

Since the natural growth of the cells or organ parts was already manifested using 3D growth chambers, animals are now needed only for the manifestation of an effect of the efficacy of the active substance to be tested that was established with the inventive method. It may even be possible to dispense with an animal control group, so that significantly fewer animals will be needed in the future for pharmacological efficacy studies.

Furthermore, the correlation between animals (mice, rats) and human cells needs to be established only once to document conclusions about the efficacy or side effects of a preparation for humans. This is currently not possible.

By using CCD cameras in the inventive apparatus, it is even possible to determine the site of the promoter-dependent reaction, i.e. if, for example, a bone cross-section is added at a certain point in time to a prostate carcinoma, the time can be established at which the prostate cancer attacks the bone marrow, or what active substances prevents this. This is currently not possible with human cells.

### BRIEF DESCRIPTION OF THE DRAWINGS

Two exemplary embodiments of the inventive apparatus will now be described in detail in conjunction with figures, in which:

FIG. 1 is a schematic longitudinal cross-sectional view of a first exemplary embodiment,

FIG. 2 is a schematic longitudinal cross-sectional view of a second exemplary embodiment.

### DETAILED DESCRIPTION OF THE INVENTION

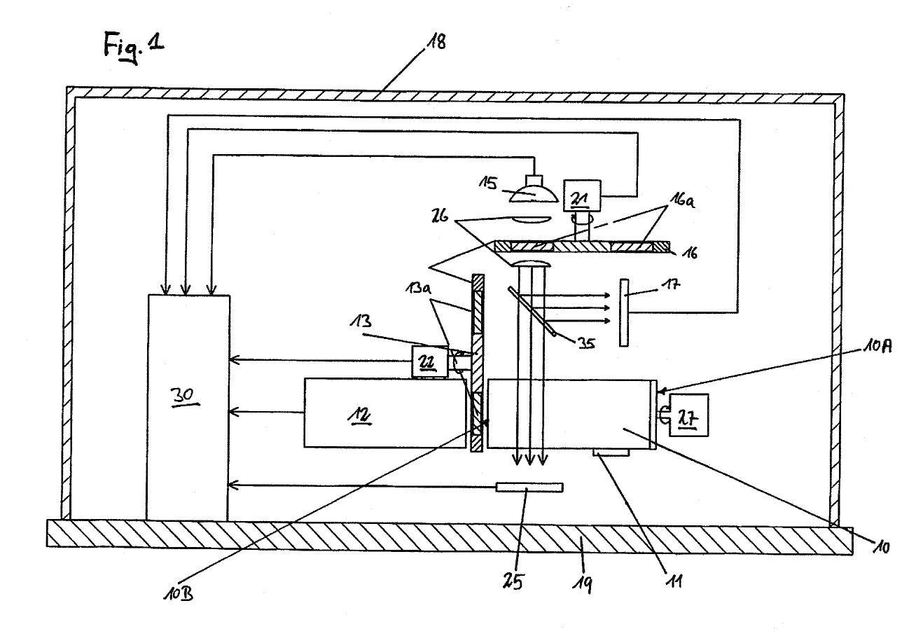

FIG. 1 schematically shows a first exemplary embodiment of the invention.

The apparatus shown in FIG. 1 has a 3D growth chamber 10, a photomultiplier 12, a light source in the form of a halogen lamp 15, two photodiodes 17 and 25, a semitransparent mirror 35, and collimator lenses 26. 3D growth chamber 10 is a hollow cylinder of a transparent material such as, for example, glass. Coupled to the first front face 10A of 3D growth chamber is an electric motor 27 connected to set 3D growth chamber into rotation about its longitudinal axis, which is horizontal in the view of FIG. 1. The inlet window of photomultiplier 12 is directed to face the second front face 10B of 3D growth chamber. 3D growth chamber 10 has, for example on its outer face, a closeable feed opening 11 serving as a feed device for adding cell cultures, growth media and active substances. The above-described components are fully enclosed, namely on the underside by an opaque base plate 19 and by an opaque hood 18 that is placed on base plate 19 so as to be light-tight.

The described apparatus is suitable for performing bioluminescence or biofluorescence measurements on cells or organ parts (also human) that grow in 3D growth chamber 10. This is done by first adding, through closeable feed opening 11, cells/organ parts, a liquid growth medium, and the active substance that is to be studied. The cells/organ parts that grow in 3D growth chamber 10 contain transfected cells that exhibit bioluminescence or biofluorescence. Afterwards hood 18 is placed onto base plate 19.

When performing a bioluminescence measurement, halogen lamp 15 must remain turned off. The bioluminescence photons that exit from the second front face 10B of 3D growth chamber 10 enter photomultiplier 12 and are converted there in a known manner into electrical signals that are counted by means of conventional evaluation electronics 30.

In BRET (Bioluminescence Resonance Energy Transfer) measurements, an emission filter motor 22 drives an emission filter wheel 13 and an appropriate emission filter 13a is placed between the second front face 10B of 3D growth chamber 10 and the inlet window of photomultiplier 12. A first measurement is performed as described above using the photomultiplier. Afterwards, after turning the emission filter wheel 13, a second measurement is performed with another filter 13a having a different transmission wavelength. Afterwards the signal ratio between the two measurements is determined.

If a biofluorescence measurement is to be performed, halogen lamp 15 is switched on, so that light is emitted through an excitation filter 16a into 3D growth chamber 10. As a rule, multiple excitation filters 16a are available that are arranged in an excitation filter wheel 16 that can be rotated by means of an excitation filter motor 21. The excitation light travels via the described light path into 3D growth chamber 10, where it excites biofluorescence. Since the intensity of the biofluorescence depends not only on the properties of the material in which biofluorescence is induced, but also on the intensity of the excitation light, a semitransparent mirror 35 is provided in the light path of the excitation light that reflects half (or another known percentage) of the emission light onto a first photodiode 17, so that a corresponding reference signal is generated to allow a constant excitation to be maintained.

The excitation light may additionally be used for measuring the cell density or organ density turbidimetrically. This purpose is served by a second photodiode 25 that is disposed on the optical axis of the excitation light, at the opposite side of chamber 10 from lamp 15, lenses 26 and mirror 35. The excitation light from lamp 15 travels through 3D growth chamber 10 perpendicular to the longitudinal axis of chamber 10. The two collimator lenses 26 serve to collimate the excitation light.

The second front face 10B of growth chamber 10 may be lens-shaped in order to optimally direct photons to the detector. If a photomultiplier is used as the light detector, the outer face of 3D growth chamber 10 is preferably mirror-coated at least in sections, in order to increase the photon yield. Since it is not possible, anyway, to achieve a spatial resolution using a single photomultiplier, the mirror-coating does not produce any shortcomings. In cases in which a biofluorescence measurement is to be performed, of course, no mirror-coating may be provided in the region in which light is emitted into 3D growth chamber 10, as the excitation light cannot be coupled-in otherwise. In cases in which a spatial resolution is to be attained, a CCD camera, which, as a rule, is also cooled like a photomultiplier, may be used in lieu of a photomultiplier. In this case, mirror-coating of 3D growth chamber 10 should be dispensed with, as it conflicts with attaining spatial resolution.

The entire light measuring unit is designed so as to be foldable toward the rear, so that growth chamber 10 is easily accessible, e.g. for flange-mounting, collection, adding of components. Since the location of PMT detector unit 12 is always the same, the growth chamber must always have the same length for different volumes.

The space that is enclosed by hood, or housing, 18 and base plate 19 is heated by means of a controllable heater, since the interior of the 3D growth chamber is to be at a constant temperature of 37° C.±0.5° C., but direct heating of the 3D growth chamber is generally not desired. Photomultiplier 12, or the CCD camera that is provided in lieu of photomultiplier 12, on the other hand, is cooled to minimize the noise component in the generated signal.

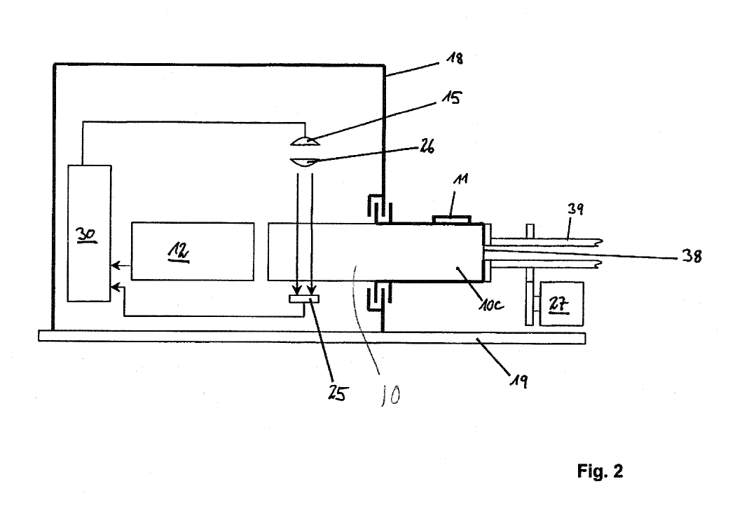

FIG. 2 shows a second exemplary embodiment. In contrast to the first exemplary embodiment, here 3D growth chamber 10 is not located completely within light-tight hood 18, but has an end 10C that projects out of the same via a light seal 32. This has the advantage that closeable opening 11 of the 3D growth chamber is accessible also immediately prior to and during the measuring process, which must be performed in the dark. If a substance is to be added into the interior of the 3D growth chamber during the measuring process, a light-tight sluice may optionally be provided at the closeable opening 11, in order to prevent any light incidence.

An arrangement of this kind additionally requires that the rearward end 10C of 3D growth chamber 10 that projects out of the housing formed by hood 18 and base plate 19 is provided with a light-tight coating. In this case it is useful to first apply onto the glass cylinder that, as a rule, forms the wall of the 3D growth chamber, a reflective surface (if desired) and then a light-tight, or opaque, layer, for example in the form of black paint. It is furthermore useful to thermally insulate the end 10C that projects out from the housing, in order to guarantee a uniform temperature in the interior of the 3D growth chamber.

The mode of functioning of this embodiment is as described above; specifically, the same optical instruments, light sources and sensors may be provided, although not all of them are shown here.

It is furthermore possible, alternatively or in addition to the closeable opening 11, to provide in the first front face 10A of the 3D growth chamber, preferably concentrically to the longitudinal axis of the cylindrical 3D growth chamber, a feed device in the form of an opening that is closed by a diaphragm 38. For this purpose, the drive shaft 39 that connects the 3D growth chamber to electric motor 27 via a gear train is designed hollow so that diaphragm 38 is accessible from the outside. In this manner, active substances, for example, can be added through an opening in this diaphragm into the interior of the 3D growth chamber without having to interrupt the rotation of the 3D growth chamber. A feed line (not depicted) may be arranged for this purpose within hollow drive shaft 39.

All of the operating conditions and parameters for carrying out measuring operations according to the invention may be selected according to principles already known in the art.

This application relates to subject matter disclosed in German Application Number DE 10 2008 010 436.1, filed 21 Feb. 2008, the disclosure of which is incorporated herein by reference.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

### LIST OF REFERENCE NUMERALS

10 3D growth chamber
10A first front face
10B second front face
10C end projecting out of the housing
11 feed opening
12 photomultiplier
13 emission filter wheel
13a emission filter
15 light source
16 excitation filter wheel
16a excitation filter
17 first photodiode
18 housing
19 base plate
20 growth chamber motor
21 excitation filter motor
22 emission filter motor
25 second photodiode
26 collimator lenses
27 electric motor
30 evaluation electronics
32 light seal
35 semitransparent mirror
38 diaphragm
39 drive shaft

What is claimed is:

1. An apparatus for measuring photon emissions from marked cells, said apparatus comprising:
a rotatable, at least partly transparent growth chamber/bioreactor (10) for accommodation of cell cultures, growth media and active substances, said chamber/bioreactor comprising at least one feed device for introducing the cell cultures, growth media and active substances into said chamber/bioreactor;
a light detector positioned for detection of photons emitted over an emission path from marked cells in said growth chamber/bioreactor; and
a light-tight enclosure enclosing at least the emission path for the photons from said growth chamber/bioreactor to said light detector.

2. The apparatus according to claim 1, further comprising mechanisms for excitation of fluorescence in the marked cells.

3. The apparatus according to claim 2, further comprising: a light source (15) disposed for directing light into said growth chamber/bioreactor; an excitation filter (16a) interposed between said light source and said growth chamber/bioreactor; and a photodiode (25) disposed to receive light from said light source passing through said growth chamber/bioreactor.

4. The apparatus according to claim 1, further comprising mechanisms for performing absorption measurements.

5. The apparatus according to claim 1, wherein said light detector comprises a photomultiplier.

6. The apparatus according to claim 5, wherein: said chamber/bioreactor has a transparent front face (10B); and said apparatus further comprises an emission filter (13a) disposed between said photomultiplier (12) and the transparent front face (10B) of said growth chamber/bioreactor.

7. The apparatus according to claim 1, wherein said light detector comprises a CCD camera

8. The apparatus according to claim 1, said light-tight enclosure is a light-tight hood (18) in which said growth chamber/bioreactor and said light detector are housed.

9. The apparatus according to claim 7, wherein: said growth chamber/bioreactor is a 3D growth chamber; one end (10C) of said growth chamber extends out of said hood and is light-tight; and said apparatus further comprises a light seal (32) associated with said hood and through which said one end of said growth chamber extends.

10. The apparatus according to claim 8, further comprising at least one feed device disposed at said one end (10C) of said 3D growth chamber that projects out from said hood.

11. The apparatus according to claim 1, said growth chamber/bioreactor has an outer wall at least a portion of which is reflecting.

12. The apparatus according to claim 1, said growth chamber/bioreactor comprises at least one feed device having an opening, and a diaphragm (38) covering said opening.

13. The apparatus according to claim 11, wherein: said growth chamber/bioreactor is a 3D growth chamber constituted by a hollow cylinder; and said opening that is covered by said diaphragm is disposed on a front face (10A) of said hollow cylinder.

14. A method for emission measurement of transfected cells comprising the following process steps:
providing the apparatus according to claim 1;
introducing the transfected cells into the growth chamber/bioreactor;
monitoring growth of the transfected cells in the growth chamber/bioreactor;
introducing an active substance that is to be tested into the growth chamber/bioreactor;
measuring photons emitted from the growth chamber/bioreactor, continually or in specifiable time intervals; and
displaying and/or storing a representation of the time progression of the intensity of the measured photons.

15. The method according to claim 13, further comprising performing an absorption measurement to adjust for cell density.

16. The method according to claim 13 carried out in the pursuit of pharmacokinetics.

17. The method according to claim 13, carried out for determining the effect of a given active substance on other cells for the detection of side effects.
